SEQUENCE LISTING (4 sequences)

SEQ ID NO: 1 - DNA, 20 bp, Artificial Sequence
  Synthetic primer
tctccaacacccactaccga

SEQ ID NO: 2 - DNA, 23 bp, Artificial Sequence
  Synthetic primer
agcggtcttctaccttactcccc

SEQ ID NO: 3 - DNA, 20 bp, Artificial Sequence
  Synthetic primer
caaggccaaccgcgagaaga

SEQ ID NO: 4 - DNA, 22 bp, Artificial Sequence
  Synthetic primer
ccagaggcgtacagggatagca

(19) United States
(12) Patent Application Publication
(10) Pub. No.: US 2016/0030539 A1
(43) Pub. Date: Feb. 4, 2016

(54) CANCER TESTIS ANTIGEN SPERM PROTEIN 17 AS A TARGET FOR BREAST CANCER IMMUNOTHERAPY AND DIAGNOSIS

(71) Applicant: Texas Tech University System, Lubbock, TX (US)

(72) Inventor: Maurizio Chiriva-Internati, Lubbock, TX (US)

(21) Appl. No.: 14/815,330

(22) Filed: Jul. 31, 2015

Related U.S. Application Data
(60) Provisional application No. 62/032,250, filed on Aug. 1, 2014.

(51) Int. Cl. A61K 39/00; A61K 9/127; G01N 33/574; A61K 45/06

(52) U.S. Cl. CPC A61K 39/0011; A61K 45/06; A61K 9/127; G01N 33/57415; A61K 2039/5154; A61K 2039/5158; G01N 2333/705; G01N 2800/7028; G01N 2800/52

(57) ABSTRACT

The present invention includes compositions and methods for treating a breast cancer cell, a metastatic breast cancer cell, or a triple negative breast cancer cell comprising: identifying a subject in need for treatment for a breast cancer cell, a metastatic breast cancer cell, or a triple negative breast cancer cell; and administering a therapeutically effective amount of a formulation that leads to the presentation of an immunogenic SP17 protein or peptide antigen on an antigen presenting cell to activate T cells that are SP17-specific T cells, wherein the SP17-specific T cells impair the growth of the breast cancer cell, the metastatic breast cancer cell, or the triple negative breast cancer cell growth.

### CROSS-REFERENCE TO RELATED APPLICATIONS

This application claims priority based on U.S. Provisional Application No. 62/032,250, filed Aug. 1, 2014. The contents of which is incorporated by reference in its entirety.

### TECHNICAL FIELD OF THE INVENTION

The present invention relates in general to the field of cancer immunotherapy and diagnosis, and more particularly, to the identification of cancer testis antigen sperm protein 17 (SP17) as a new target for breast cancer immunotherapy and diagnosis.

### STATEMENT OF FEDERALLY FUNDED RESEARCH

None.

### INCORPORATION-BY-REFERENCE OF MATERIALS FILED ON COMPACT DISC

The present application includes a Sequence Listing which has been submitted in ASCII format via EFS-Web and is hereby incorporated by reference in its entirety. Said ASCII copy, created on ______, 2015, is named TECH1114_SeqList and is ______ kilobytes in size.

### BACKGROUND OF THE INVENTION

Without limiting the scope of the invention, its background is described in connection with breast cancer therapies.

According to the latest data available from the United States Cancer Statistics (USCS, http://apps.nccd.cdc.gov/uscs) 186,467 women were diagnosed with breast carcinoma (BC) and 41,116 died from the disease in 2005. The most common breast cancers are invasive ductal breast carcinoma (IDBC) and invasive lobular carcinoma (ILC) [1,2]. However, IDBC accounts for 80% of all cases, while ILC affects only about 5% to 10% of all patients [2]. Further, the risk of mortality of women with ILC is 26% lower than women with IDBC [3].

Among BC treatment options, anti-estrogen drugs (tamoxifen or aromatase inhibitors), and epidermal growth factor-targeted therapies (trastuzumab) have largely proven effective [4-6]. But even if these therapies are still the mainstay of breast cancer treatment, they display important disadvantages. Firstly, tamoxifen and aromatase inhibitors increase the risk of endometrial cancer and osteoporosis, respectively [7], while anti-epidermal growth factor agents cause severe cardiac toxicity [8]. Secondly, the efficacy of endocrine-active drugs is limited by intrinsic and acquired resistance [9]. Finally, these approaches are useless in triple-negative breast cancers (TNBC) that do not express estrogen, progesterone or human epidermal growth factor receptors and are characterized by an extremely poor prognosis [10]. These factors underline the urgent need for novel clinical strategies.

The most promising approach is based on cancer vaccines. Such vaccinations exploit tumor-associated antigens able to raise specific cytotoxic T lymphocyte (CTL) antitumor responses both in animal models and in clinical settings [11-14]. Targets for ideal immunotherapy should have an expression pattern highly restricted to tumor cells to prevent autoimmunity. The main categories of antigens that meet this requirement are mutated, viral and differentiation antigens. Numerous clinical trials are currently ongoing for breast cancer vaccines based, for instance, on MUC-I or estrogen receptors (clinical trial identifiers NCT00986609 and 6 NCT00343109). More recently, a novel and expanding group of tumor-associated antigens has been discovered: since their expression is testis-restricted, but strongly associated with many tumors, they are called cancer/testis antigens (CTA). Their marked immunogenicity and restricted expression make them ideal targets for immunotherapies: CTA vaccinations have been successfully performed in patients with solid cancers of different origins such as lung, ovary and melanoma [15-17]. CTA expression in BC has been found to affect cell proliferation [18,19] and to be correlated with a lack of estrogen receptor expression and poor prognosis [20,21]. However a need remains for novel therapeutics that target breast cancers, in particular the cancers that are hardest to treat because they have become refractory to standard chemotherapeutic agents.

### SUMMARY OF THE INVENTION

In one embodiment, the present invention includes a method for treating a breast cancer cell, a metastatic breast cancer cell, or a triple negative breast cancer cell comprising: identifying a subject in need for treatment for a breast cancer cell, a metastatic breast cancer cell, or a triple negative breast cancer cell; and administering a therapeutically effective amount of a formulation that leads to the presentation of an immunogenic SP17 protein or peptide antigen on an antigen presenting cell to activate T cells that are SP17-specific T cells, wherein the SP17-specific T cells impair the growth of the breast cancer cell, the metastatic breast cancer cell, or the triple negative breast cancer cell growth. In one aspect, the formulation further comprises an immunogenic SP17 protein or peptide antigen and an adjuvant. In another aspect, the formulation is defined further as an antigen presenting cell that has been pre-loaded with the isolated immunogenic SP17 protein or peptide. In another aspect, the formulation is defined further as an antigen presenting cell that expresses an immunogenic SP17 protein or peptide and the antigen presenting cell is defined further as an autologous dendritic cell. In another aspect, the isolated immunogenic SP17 protein or peptide antigen comprises a vaccine composition. In another aspect, the method further comprises the steps of: obtaining a sample from the subject; and determining the presence or absence of a specific anti-SP17 immunoglobulins in the sample, wherein the presence of the specific anti-SP17 immunoglobulins in the sample indicating the subject is afflicted with or at least at risk of developing breast cancer. In another aspect, the sample is a blood sample. In another aspect, the method further comprises obtaining naïve T cells from the subject, activating the naïve T cells in the presence of antigen presenting cells that present immunogenic SP17 under conditions that activate the T cells. In another aspect, the method further comprises obtaining naïve T cells from the subject, activating the naïve T cells in the presence of antigen presenting cells that present immunogenic SP17 under conditions that activate the T cells in vitro and then introducing the activated T cells into the subject. In another aspect, the triple negative breast cancer is resistant to chemotherapeutic agents. In another aspect, the SP17 protein or peptide is loaded into a liposome for delivery into the antigen presenting cell, the SP17 protein or peptide is expressed in the antigen presenting cell by transient or stable transfection or the SP17 peptide is loaded directly onto MHC on the surface of the antigen presenting cell.

Another embodiment of the present invention includes a method of treating a breast cancer cell, a metastatic breast cancer cell, or a triple negative breast cancer in a human subject comprising the steps of: identifying a human subject in need of treatment for breast cancer cell, a metastatic breast cancer cell, or a triple negative breast cancer by detecting SP17; and administering a therapeutically effective amount of a formulation that includes an isolated antigen presenting cells that is loaded with an immunogenic SP17 protein or peptide antigen capable of activating T cells to suppress the growth of the breast cancer cell, the metastatic breast cancer cell, or the triple negative breast cancer cell growth. In one aspect, the method further comprises the step of obtaining a biological sample from the subject suspected of having SP17 and determining the present or absence of the SP17, wherein a decrease or the absence of SP17 in the biological sample following treatment is indicative that the treatment if effective. In another aspect, the antigen presenting cells is defined further as a dendritic cell. In another aspect, the sample is a blood sample. In another aspect, the method further comprises obtaining naïve T cells from the subject, activating the naïve T cells in the presence of antigen presenting cells that present immunogenic SP17 under conditions that activate the T cells. In another aspect, the method further comprises obtaining naïve T cells from the subject, activating the naïve T cells in the presence of antigen presenting cells that present immunogenic SP17 under conditions that activate the T cells and then introducing the activated T cells into the subject. In another aspect, the triple negative breast cancer is resistant to chemotherapeutic agents. In another aspect, the tumor cell or breast cancer is resistant tamoxifen. In another aspect, the method further comprises the step of administering the therapeutic agent before, after, or concurrently as a combination therapy with one or more active agents that target at least one of breast cancer cells, metastatic breast cancer cells, or triple negative breast cancer cells. In another aspect, the triple negative breast cancer is resistant to chemotherapeutic agents. In another aspect, the method further comprises the step of determining if the subject no longer responds to one or more chemotherapeutic agents prior to administering the antigen presenting cells that present SP17 to T cells.

Yet another embodiment of the present invention includes a method of treating a cancer resistant to chemotherapy comprising the steps of: identifying a subject having a breast cancer resistant to the chemotherapeutic agents or in need of increased effectiveness of the one or more chemotherapeutic agents, wherein the breast cancer is a triple negative breast cancer; and administering a therapeutic agent carrier to the subject in an amount sufficient to suppress or inhibit the growth of breast cancer cells that express SP17 leading to an apoptosis, an arrested proliferation, or a reduced invasiveness of the breast cancer cells. In one aspect, the subject is further provided one or more chemotherapeutic agents comprise platinum drugs, carboplatin, tamoxifen, ER antagonists, or any combinations thereof. In another aspect, the inhibitor of SP17 is administered before, after, or concurrently as with the one or more chemotherapeutic agents. In another aspect, the breast cancer cells are triple negative breast cancer resistant to chemotherapeutic agents. In another aspect, the subject is a human.

Another embodiment of the present invention includes a method for diagnosing breast cancer progression, prognosis, and resistance: obtaining a biological sample from a patient suspected of having breast cancer; detecting SP17 in the biological sample, wherein the presence of SP17 is indicative of the patient having breast cancer; and administering a therapeutic agent that targets cells expressing SP17 in an amount effective to reduce the number of breast cancer cells in the patient. In another aspect, the therapeutic agent that targets cells expressing SP17 is provided before, after, or concurrently as a combination therapy with one or more chemotherapeutic agents that target at least one of breast cancer cells, metastatic breast cancer cells, or triple negative breast cancer cells.

### BRIEF DESCRIPTION OF THE DRAWINGS

For a more complete understanding of the features and advantages of the present invention, reference is now made to the detailed description of the invention along with the accompanying figures and in which:

FIG. 1 is a gel that shows RT-PCR for SP17 in BC cell lines MD-MB-3681 (MD-MB), HCC70 and non-tumoral 184B5 cells. Testis-derived total RNA was used as a positive control. A reaction without cDNA template (no temp) and with RT reaction performed without retro-transcriptase (no RT) were included as negative controls. β-actin was used as an internal control for all PCR reactions.

FIG. 2 is a gel that shows an analysis of SP17 expression in the indicated normal tissues. Testis-derived total RNA was used as a positive control, while reaction without cDNA template (no temp) or with RT reaction performed without retro-transcriptase (no RT) were included as negative controls. β-actin was used as an internal control for all PCR reactions.

FIG. 3 is a Western blot for SP17 in HCC70 and MDA-MB-361 BC cell lines and 184B5 non-tumoral cells. Bovine-serum albumin (BSA) was used as a negative control, while human SP17 recombinant protein was used as a positive control.

FIG. 4 is an RT-PCR for SP17 in patients' samples. Testis-derived total RNA was used as a positive control, while reaction without cDNA template (no temp) or with RT reaction performed without retro-transcriptase (no RT) were included as negative controls. β-actin was used as an internal control for all PCR reactions.

FIGS. 5A to 5L1-5L4 are micrographs that show the immunohistochemistry for SP17 protein expression in the indicated tissue types. Pictures are representative of all 29 analyzed primary samples. (FIGS. 5A to 5L1-5L4). FIG. 5M is a graph that shows the presence of circulating SP17-specific auto-antibodies.

FIG. 6 is a graph that summarizes an ELISA showing the presence of IgG anti-SP17 in 10/22 BC patients (45%). The cut-off point (red line, mean+3 STDEV), based on control values, was OD₄₅₀ nm=0.1645. 23.

FIG. 7 is a graph that shows successful generation of anti-SP17 cytotoxic T-lymphocytes (CTLs). SP17-protein propagated CTLs generated from 10 BC patients were able to kill autologous BC cells. Experiments were performed in triplicate (error bars represent SD; E=effector cells, T=target cells).

FIG. 8 is a graph that shows an analysis of SP17-stimulated T-cells specificity. SP17-stimulated T-cells were able to kill autologous lymphoblastoid cells (LCL) in a SP17-dependent manner when loaded with rSP17 protein as a positive control. T cells were unable to kill LCL alone or LCL pulsed with HPV16-E6 antigen as negative controls. Target cell lysis was inhibited by an antibody directed at monomorphic HLA class I molecules but not HLA class II molecules (E:T ratio was 20:1 for all analyses).

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The inventors have discovered a novel cancer testis antigen (CTA), SP17, in breast cancer that is both a biomarker and an immunotherapy target. It is shown herein that targeting SP17 is effective for the treatment of primary, metastatic and triple negative breast cancer cells, SP17 expression is completely absent in normal breast tissue and a wide panel of normal tissues. The highly restricted expression pattern makes SP17 an excellent candidate for a cancer vaccine target. The inventors have further shown the efficacy of immunotherapeutic approaches by generating autologous SP17-specific T cells in vitro and demonstrate that they efficiently and specifically kill the SP17 expressing breast cancer cells. It is further shown herein that SP17 is a biomarker of breast cancer progression, prognosis, resistance and treatment.

In summary, the invention represents a novel breast cancer immunotherapy that is useful in the treatment of normal, metastatic, and triple negative breast cancers. The invention is an improvement over current therapies that are largely ineffective against metastatic and triple negative breast cancers. Additionally, cancer vaccines work through naturally exploiting the human body's immune system and raising specific cytotoxic T lymphocyte anti-tumor responses which may avoid some of the health risks associated with traditional chemotherapeutics.

The cancer/testis antigen Sperm Protein 17 (SP17) is a highly conserved mammalian protein, normally found in spermatozoa, where it contributes to sperm maturation, capacitation and acrosomal reaction [22], (GenBank Accession No. AF334735 (nucleic acid), AAK20878.1 (amino acid), which sequences are incorporated herein by reference). Aberrant SP17 expression has been shown in ovarian and esophageal cancers [23,24], nervous system tumors [25] and in multiple myeloma [26]. SP17 mechanisms of action in cancer cells are still poorly understood. Nonetheless, it has been shown that SP17 mediates cell-cell adhesion in malignant B-lymphocytes through heparan-sulfate [27], and enhances cell motility and drug resistance in ovarian cancer [28]. Thus, SP17-targeted immunotherapy is likely to prove effective in various solid and hematological tumors.

To date, SP17 expression in BC has never been identified or investigated. Surprisingly, it was found herein that SP17 is expressed at the mRNA and protein levels in human breast cancer cell lines, primary IDBC and normal breast tissue. The present inventors demonstrate herein the use of SP17 immunogenicity for the development of breast cancer immunotherapy.

Breast carcinoma is a major health issue for millions of women. Current therapies display serious side effects, and are hardly effective in patients with metastatic and/or poorly-differentiated tumors. Thus, the need for novel therapies is urgent. Vaccinations exploiting tumor-associated antigens able to raise specific cytotoxic T lymphocyte-mediated antitumor responses are likely to become the therapy of choice in the future. Cancer/testis antigens are a group of tumor-associated antigens displaying ideal features as vaccine targets. The cancer/testis antigen SP17 has been found aberrantly expressed in different neoplasia, such as ovarian and esophageal cancers, nervous system tumors and multiple myeloma. Thus, SP17-targeted immunotherapy is expected to prove effective in various solid and hematological tumors. Here SP17 expression and immunogenicity in breast cancer is evaluated for the first time.

The inventors evaluated SP17 expression at the transcriptional and translational levels in breast cancer cell lines, normal tissues and biopsies of invasive ductal breast carcinoma and normal mammary ducts. Then, the inventors generated in vitro SP17-specific cytotoxic T lymphocytes and evaluated their capacity of lysing breast tumor cells.

The inventors show for the first time SP17 to be expressed in breast cancer cell lines and primary tumor samples. SP17 was absent in all normal breast samples analyzed and in normal tissues derived from different organs. Finally, the inventors demonstrate that SP17 is a suitable target for BC immunotherapy, as specific anti-SP17 antibodies was detected in patients' sera and the induction and/or activation of SP17-specific HLA class I-restricted cytotoxic T lymphocytes efficiently killing tumor cells in vitro.

These data demonstrate that SP17 is a novel cancer/testis antigen in breast cancer, prompting further analysis of SP17 expression as a biomarker for tumor progression and prognosis. Further, it is shown that SP17-targeted immunotherapy for breast cancer leads to subject vaccination.

According to the latest data available from the United States Cancer Statistics (USCS, apps.nccd.cdc.gov/uscs) 186,467 women were diagnosed with breast carcinoma (BC) and 41,116 died from the disease in 2005. The most common breast cancers are invasive ductal breast carcinoma (IDBC) and invasive lobular carcinoma (ILC) [1,2]. However, IDBC accounts for 80% of all cases, while ILC affects only about 5% to 10% of all patients [2]. Further, the risk of mortality of women with ILC is 26% lower than women with IDBC [3].

Among BC treatment options, anti-estrogen drugs (tamoxifen or aromatase inhibitors), and epidermal growth factor-targeted therapies (trastuzumab) have largely proven effective [4-6]. But even if these therapies are still the mainstay of breast cancer treatment, they display important disadvantages. Firstly, tamoxifen and aromatase inhibitors increase the risk of endometrial cancer and osteoporosis, respectively [7], while anti-epidermal growth factor agents cause severe cardiac toxicity [8]. Secondly, the efficacy of endocrine-active drugs is limited by intrinsic and acquired resistance [9]. Finally, these approaches are useless in triple-negative breast cancers (TNBC) that do not express estrogen, progesterone or human epidermal growth factor receptors and are characterized by an extremely poor prognosis [10]. These factors underline the urgent need for novel clinical strategies.

The most promising approach is based on cancer vaccines. Such vaccinations exploit tumor-associated antigens able to raise specific cytotoxic T lymphocyte (CTL) antitumor responses both in animal models and in clinical settings [11-14]. Targets for ideal immunotherapy should have an expression pattern highly restricted to tumor cells to prevent autoimmunity. The main categories of antigens that meet these requirements are mutated, viral and differentiation antigens. Numerous clinical trials are currently ongoing for breast cancer vaccines based, for instance, on MUC-1 or estrogen receptors (clinical trial identifiers NCT00986609 and 6 NCT00343109). More recently, a novel and expanding group of tumor-associated antigens has been discovered: since their expression is testis-restricted, but strongly associated with many tumors, they are called cancer/testis antigens (CTA). Their marked immunogenicity and restricted expression make them ideal targets for immunotherapies: CTA vaccinations have been successfully performed in patients with solid cancers of different origins such as lung, ovary and melanoma [15-17]. CTA expression in BC has been found to affect cell proliferation [18,19] and to be correlated with a lack of estrogen receptor expression and poor prognosis [20,21].

The cancer/testis antigen Sperm Protein 17 (SP17) is a highly conserved mammalian protein, normally found in spermatozoa, where it contributes to sperm maturation, capacitation and acrosomal reaction [22]. Aberrant SP17 expression has been shown in ovarian and esophageal cancers [23,24], nervous system tumors [25] and in multiple myeloma [26]. SP17 mechanisms of action in cancer cells are still poorly understood. Nonetheless, it has been shown that SP17 mediates cell-cell adhesion in malignant B-lymphocytes through heparan-sulfate [27], and enhances cell motility and drug resistance in ovarian cancer [28]. Thus, SP17-targeted immunotherapy is likely to prove effective in various solid and hematological tumors.

SP17 expression in BC has not been investigated. In the present study, the inventor evaluated SP17 expression at the mRNA and protein levels in human breast cancer cell lines, primary IDBC and normal breast tissue. Then, SP17 immunogenicity was assessed for the development of breast cancer immunotherapy.

Materials and Methods. Cell lines and primary samples. A normal breast epithelial cell line and BC cell lines were purchased from the American Type Culture Collection (ATCC, www.atcc.org): 184B5 cells (ATCC No. CRL-8799) are benzo(a)pyrene-transformed cells originated from a normal mammary epithelium, MDA-MB-361 cells (ATCC No. HTB-27) were originated from a brain metastasis of breast adenocarcinoma, while HCC70 cells (ATCC No. CRL-2315) were derived from a primary ductal carcinoma and these cells are negative for the expression of epidermal growth factor receptor-2. Cell lines were analyzed in 2 passages after purchasing, to rule out possible alterations due to in vitro propagation. Primary samples were obtained after informed consent at Texas Tech University Health Sciences Center, USA and IRCCS Istituto Clinico Humanitas, Italy, according to the Declaration of Helsinki, and consisted of needle biopsies taken from milk ducts.

RNA isolation and Reveres-Transcriptase-Polymerase Chain Reaction (RT-PCR). Total RNA was isolated from BC cell lines or biopsy materials through the Aurum Total RNA Mini Kit (Bio-Rad, Hercules, Calif.) and 1 μg was retro-transcribed using the iScript cDNA Synthesis Kit (Bio-Rad). RNAs from normal human tissues were obtained from FirstChoice® Total RNA (Ambion, Austin, Tex.). 1/20 of retro-transcription reaction volume was PCR-amplified in 20 μL reaction with iTaq hot-start DNA polymerase (Bio-Rad). Primers sequences were: SP17 left 5′-TCTCCAACACCCACTACCGA-3′ (SEQ ID NO.: 1), SP17 right 5′-AGCGGTCTTCTACCTTACTCCCC-3′ (SEQ ID NO.: 2), β-actin left 5′-CAAGGCCAACCGCGAGAAGA-3′ (SEQ ID NO.: 3), β-actin right 5′-CCAGAGGCGTACAGGGATAGCA-3′ (SEQ ID NO.: 4).

All reactions were performed at 57° C. annealing temperature for 38 cycles with 1.5 mM MgCl₂. 10 μL PCR reaction were run in 2% w/v agarose gel stained with ethidium bromide. Pictures were taken after 30 minutes run using the Molecular Imager ChemiDoc XRS+ System (Bio-Rad) equipped with a Quantity One 1-D Analysis Software (Bio-Rad).

Protein analysis. Total proteins were isolated through the ReadyPrep protein extraction kit (Bio-Rad) and quantified using the Bradford colorimetric method (Bio-Rad). 80 μg total protein extracts were subjected to electrophoresis in a mini-gel system (Bio-Rad), with a 6% stacking- and 12% resolving-polyacrylamide gel. Resolved proteins were electro-transferred overnight at 4° C. on an Immun-Blot PVDF membrane (Bio-Rad). Membranes were probed with the mouse anti-human-SP17 antibody produced by the present inventor, then with HRP-linked bovine anti-mouse IgG (Santa Cruz Biotechnology). After antibody hybridization, chemiluminescent signals were detected on a photographic sheet (Sigma-Aldrich, St. Louis, Mo.) using the Immun-Star WesternC Chemiluminescent Kit (Bio-Rad). Pictures of the developed photographic sheets were taken through the Molecular Imager ChemiDoc XRS+ System (Bio-Rad).

Immunohistochemistry. Biopsy material was embedded in paraffin and 3 μm thick-sections were prepared. Slices were exposed to the anti-SP17 primary antibody (Santa Cruz Biotechnology, 1:100 dilution in PBS/BSA 0.1%), and then incubated for 30 minutes with the HRP-linked secondary antibody (Santa Cruz Biotechnology, 1:500 dilution in PBS/BSA 0.1%) and 5 minutes with DAB (3,3′-diaminobenzidine, DAKO, Glostrup, Denmark). Pictures were taken at 10×, 20×, 40× and 63× objective magnifications using a DMI3000 B inverted microscope (Leica Microsystems GmbH, Wetzlar, Germany) and analyzed by the Leica Application Suite (LAS) software (Leica Microsystems GmbH).

Enzyme-linked immunosorbent assay. An enzyme-linked immunosorbent assay (ELISA) was performed on the sera of 22 BC and on 7 pooled healthy patients. Polystyrene 96-well flat-bottom plates were coated with 5 μg/μL SP17 recombinant protein (generated by the present inventors) and incubated overnight at 4° C. After washing and blocking with SuperBlock® buffer (Pierce, Rockford, Ill., USA), plates were placed at 37° C. for 2 hours. Each sample, as well as the negative controls (PBS supplemented with 10% FBS), were diluted 1:1000 in SuperBlock® buffer and incubated for 4 hours at RT. After washing with PBS/0.05% Tween-20, horseradish peroxidase-conjugated goat anti-human IgG (Pierce), diluted 1:5000 in SuperBlock®, was added and allowed to incubate at RT for 2 hours. Next, the 1-Step Ultra TMB-ELISA chromogenic substrate (Pierce) was added to each well for color development for 10 minutes. After blocking the reaction with sulfuric acid, the intensity was measured by the Victor2 micro plate multilabel counter (PerkinElmer, Waltham, Mass., USA) at 450 nm wavelength excitation. All samples were run in triplicate.

Isolation of peripheral blood mononuclear cells and generation of dendritic cells. Peripheral blood mononuclear cells (PBMCs) from 22 BC patients were prepared by separation of heparinized blood with density gradient centrifugation performed with Ficoll-Hypaque. PBMCs were seeded into 6-well culture plates with 3 mL RPMI 1640 supplemented with 10% fetal bovine serum (FBS) at the density of 8-10×10⁶ cells/well. After 2 hours incubation at 37° C. and 5% CO₂, non-adherent cells were removed; adherent cells were maintained in RPMI 1640 supplemented with 10% FBS, 103 IU/mL interleukin 4 (IL-4) and 800 IU/mL granulocyte-macrophage colony-stimulating factor (GM-CSF). After 1-week culture, dendritic cells (DCs) were harvested and pulsed with SP17 protein [26].

Dendritic Cell (DC) pulsing. DCs were washed twice with PBS and transferred in a 50 mL polypropylene tube. The recombinant protein (developed in the inventor's laboratory) SP17 (rSP17) was mixed with the cationic lipid DOTAP (Roche, Mannheim, Germany) at room temperature for 20 minutes, then added to the DCs for 3 hours at 37° C.

Generation of SP17-specific cytotoxic T-lymphocytes (CTLs) in vitro. Antigen pulsed DCs were co-cultured with fresh autologous PBMCs at a ratio of 1:10 in RPMI 1640 with 10% autologous serum, 10 IU/mL IL-2 and 5 ng/mL IL-7. Irradiated autologous PBMCs feeder cells and SP17 protein (50 μg/mL) were added every 7 days, while IL-2 was added every 3 days [26].

Cytotoxicity assay. A standard 4-hour Europium-release assay was performed to evaluate the cytotoxic activity of the SP17-stimulated T cells [26]. Cytotoxicity against autologous breast cancer primary cells was determined at various effector:target cell ratios in the range of 60:1 to 1:1. For the measurement of CTL-mediated lysis of normal breast duct cells (184B5), breast tumor cell lines (HCC770 and MD-MB-361) or autologous lymphoblastoid cells (LCL) alone or pulsed with HPV16-E6 antigen or with rSP1, cytotoxicity assay was performed with 20:1 effector:target ratio. To determine HLA restricted response a cytotoxicity assay was performed with or without 25 μg/mL HLA-I or HLA-II (W6/32 or L243 monoclonal antibody, respectively, BioLegend 11080 Roselle Street, San Diego, Calif.) blocking antibodies (effector:target ratio 20:1). Standard deviations were determined on the results of the experiments run in triplicates.

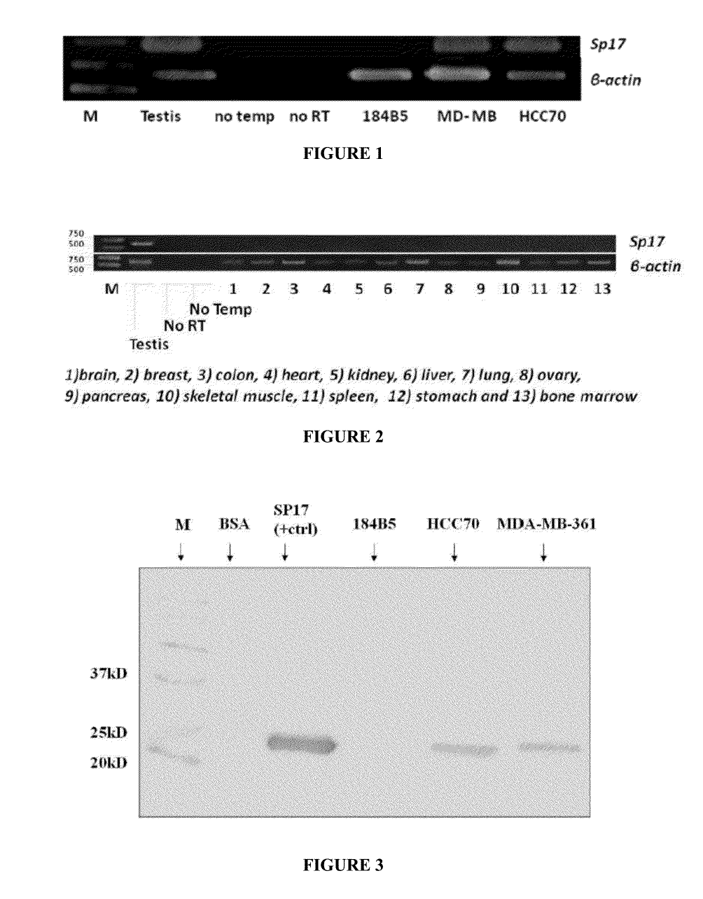

The inventors first analyzed SP17 mRNA expression in BC cell lines. As shown in FIG. 1, SP17 mRNA was detected in metastatic (MDA-MB-361) and primary (HCC70) breast cancer cell lines, but not in the normal mammary epithelium cell line 184B5. Further, it was shown that SP17 mRNA expression was absent in a panel of human normal tissues, namely brain, breast, colon, heart, kidney, liver, lung, ovary, pancreas, skeletal muscle, spleen, stomach and bone marrow (FIG. 2). The inventors further validated the data by analyzing SP17 expression at the protein level in 184B5, HCC70 and MDA-MB-361 cell lines by Western blot: FIG. 3 shows that SP17 protein is expressed in BC cell lines but not in 184B5 non-tumor cells.

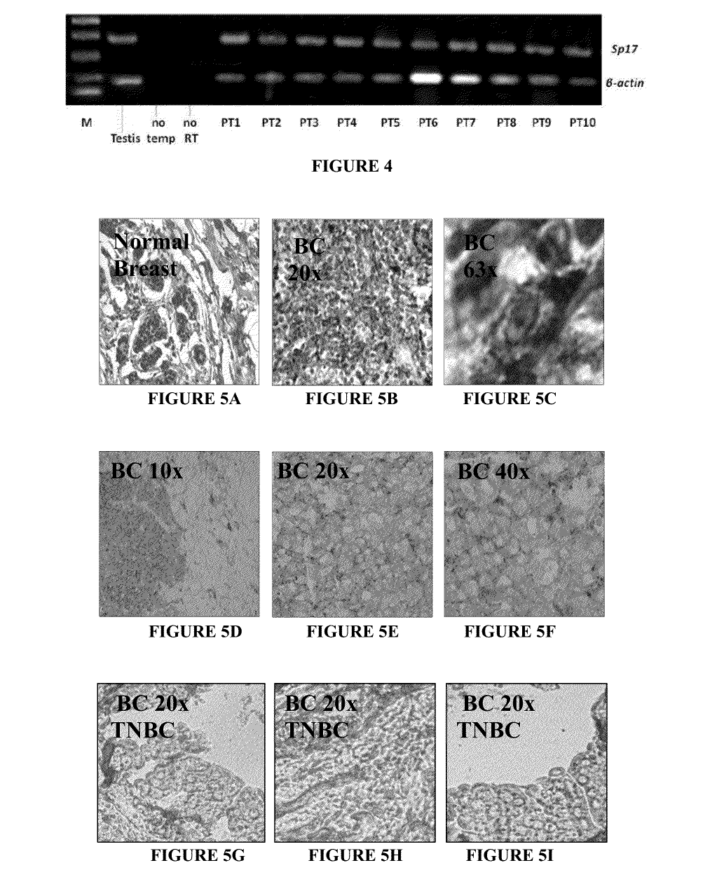

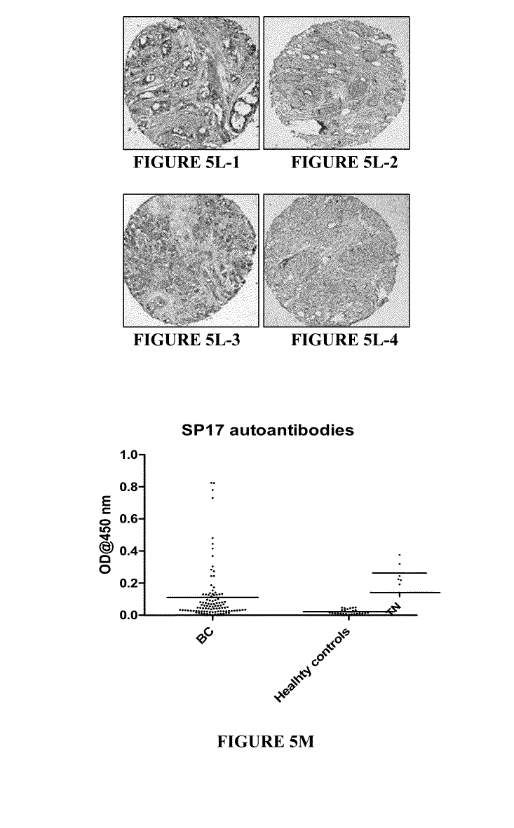

Next, the inventors analyzed SP17 expression by RT-PCR in primary cells. Samples consisted of 7 normal breast tissues and 22 primary invasive ductal BC (FIG. 4). No SP17 expression was detected in normal mammary ducts, but it was present in 10 BC samples. Results and subjects' characteristics are summarized in Table 1. SP17 expression data obtained by RT-PCR in primary samples were confirmed by immunohistochemistry: FIGS. 5A to 5L shows representative pictures taken from immunohistochemical analysis for SP17 protein in normal and tumor mammary duct cells. Positive staining was detectable in tumor samples only. FIG. 5M is a graph that shows the presence of circulating SP17-specific auto-antibodies.

FIGS. 5A to 5L shows the expression and immunogenicity of SP17 in breast cancer (BC) population including triple-negative breast cancers. FIG. 5A Control. FIG. 5B, C, D, E, F, G, H, I are representative IHC pictures of BC and TNBC samples stained with anti-SP17 antibody (brown signal indicates SP17-positive cells). FIGS. 5L-1 to 5L-4 show tissue microarrays IHC for SP17: 5L-1 and 5L-3 and breast cancer: 5L-2 and 5L-4. FIG. 5M is a graph that shows circulating anti-SP17 IgG were detected in the serum of the majority of BC patients, were not present in the serum of age-matched healthy women (healthy controls), and were elevated in triple negative (TN) BC patients.

TABLE 1
Sample characteristics and SP17 mRNA expression status.

| Tissue type | Median age | SP17 mRNA |
|---|---|---|
| Normal breast ducts (7)^{a} | 40 (18-66)^{b} | 0 (0) ^{c} |
| IDBC (22) | 48 (34-62) | 10 (45) |

^{a}number of samples;
^{b}years (range);
^{c} number of positive sample (%)

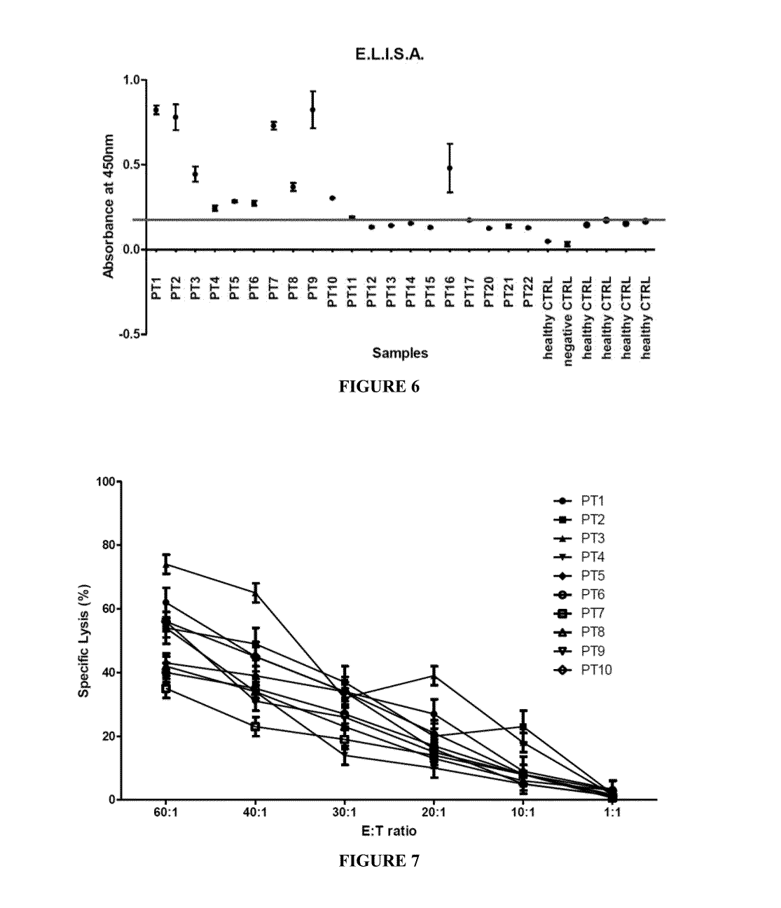

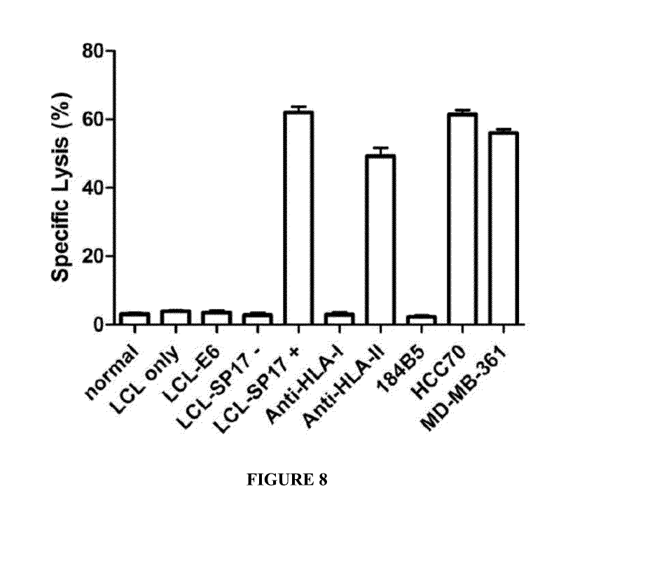

In order to evaluate the suitability of SP17 as a target for BC immunotherapy, the presence of anti-SP17 antibodies in the serum of patients' and healthy controls by ELISA technique (FIG. 6) was assayed. A positive signal was detected in 10 patients (45%). The cut-off point (mean+3 SD), determined by the healthy controls, was significantly low (0.04875OD), while negative controls had an OD425 nm=0.03235. Finally, the inventors successfully generated anti-SP17 lymphocytes from PBMCs of BC patients. Autologous SP17-stimulated T cells were able to specifically kill BC cells derived from patients' primary samples (FIG. 7). To determine the SP17-specificity of cell lysis, T cell-mediated killing of normal breast duct cells, 184B5, HCC770, MD-MB-361 cell lines or of autologous lymphoblastoid cells (LCL) alone or pulsed with HPV-E6 antigen or with rSP17 was measured. Results (FIG. 8) show that significant lysis is detectable only in SP17 expressing target cells, namely LCL-SP17, HCC70 and MD-MB-361. It is further demonstrated herein that SP17-stimulated T cells were unable to kill SP17-expressing target cells when HLA-I molecules were blocked through a specific antibody, while killing activity was not affected by HLA-II blocking (FIG. 8).

The present inventors demonstrate for the first time that SP17 is a novel cancer/testis antigen in breast cancer. It is shown herein that SP17 is expressed at both mRNA and protein levels in breast cancer cell lines, but not in normal breast-derived cells (184B5). Importantly, SP17 is present in both BC primary (HCC70) and metastatic (MDA-MB-361) cells, indicating that its expression is conserved during the metastatic spread of primary tumors. The specificity of this analysis was confirmed by the lack of SP17 detection in high-grade total RNA isolated from a wide panel of human normal tissues of different subjects, and by the positive signal displayed in testis-derived RNA. Finally, the present inventors describe for the first time the presence of SP17 mRNA protein in primary invasive ductal BC through RT-PCR and immunohistochemistry; the same analyses failed to reveal SP17 expression in all normal breast tissues analyzed, indicating the robust specificity of SP17 expression.

These data demonstrate that SP17 is a novel target for BC, as it has been successfully proven in animal models of ovarian cancer [29]. Further, SP17 expression has recently been shown to predict cisplatin resistance in esophageal squamous cancer cell lines [30]. It is shown herein that SP17 is an indicator of BC progression, prognosis and risk of drug resistance. The current need and challenge in the therapy of BC is the discovery of novel tumor-associated antigens for the development of anti-tumor vaccines [31]. It was found that SP17-restricted expression in tumor cells is a powerful target for immunotherapy, as shown in ovarian cancer [32] and multiple myeloma [33]. The present inventors detected significant levels of anti-SP17 antibodies in the serum of 45% patients with BC, proving its immunogenicity in vivo. As shown herein, the inventors were able to generate autologous SP17-specific T cells in vitro, efficiently killing SP17-expressing primary tumor cells and SP17-positive BC cell lines, but not SP17-negative control cells. Further, it is shown that cytotoxic activity of autologous SP17-activated T cells was HLA-I restricted, indicating that the anti-tumor response was mediated by CD8+ cytotoxic lymphocytes (CTL). Remarkably, these results show for the first time that targeting SP17 is an innovative immunotherapeutic approach in the cure of BC, which overcomes the limitations of current therapies.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1. Margolese R G, Hortobagyi G N, Buchholz T A. Histologic Types. In: Kufe D W, Pollock R E, Weichselbaum R R, Bast, R C Jr, Gansler, T S, Holland, J F, Frei III E, editors. Neoplasms of the Breast. Hamilton (Canada): BC Decker Inc.; 2003. p. 121.
2. Dian D, Herold H, Mylonas I, Scholz C, Janni W, Sommer H, et al. Survival analysis between patients with invasive ductal and invasive lobular breast cancer. Arch Gynecol Obstet 2009; 279(1):23-8.
3. Li C I, Moe R E, Daling J R. Survival analysis between patients with invasive ductal and invasive lobular breast cancer. Arch Gynecol Obstet 2009; 279(1):23-8.
4. Hughes-Davies L, Caldas C, Wishart G C. Tamoxifen: the drug that came in from the cold. Br J Cancer 2009; 101(6):875-8.
5. Cleator S J, Ahamed E, Coombes R C, Palmieri C, Cleator S J, Ahamed E, et al. A 2009 update on the treatment of patients with hormone receptor-positive breast cancer. Clin Breast Cancer 2009; 9 Suppl 1:S6-S17.
6. Spector N L, Blackwell K L. Understanding the Mechanisms Behind Trastuzumab Therapy for Human Epidermal Growth Factor Receptor 2-Positive Breast Cancer. J Clin Oncol 2009 Nov. 2.
7. Lin M L, Fukukawa C, Park J H, Naito K, Kijima K, Shimo A, et al. Involvement of G-patch domain containing 2 overexpression in breast carcinogenesis. Cancer Sci 2009; 100(8):1443-50.
8. de Azambuja E, Bedard P L, Suter T, Piccart-Gebhart M. Cardiac toxicity with anti-HER-2 therapies: what have we learned so far? Target Oncol 2009; 4(2):77-88.
9. Musgrove E A, Sutherland R L. Biological determinants of endocrine resistance in breast cancer. Nat Rev Cancer 2009; 9(9):631-43.
10. Arslan C, Dizdar O, Altundag K. Pharmacotherapy of triple-negative breast cancer. Expert Opin Pharmacother 2009; 10(13):2081-93.
11. Inoda S, Hirohashi Y, Torigoe T, Nakatsugawa M, Kiriyama K, Nakazawa E, et al. Cep55/c10orf3, a tumor antigen derived from a centrosome residing protein in breast carcinoma. J Immunother 2009; 32(5):474-85.
12. Sørensen R B, Svane I M, Staten P T, Andersen M H. A surviving specific T-cell clone from a breast cancer patient display universal tumor cell lysis. Cancer Biol Ther 2008; 7(12):1885-7.
13. Tsuruma T, Iwayama Y, Ohmura T, Katsuramaki T, Hata F, Furuhata T, et al. Clinical and immunological evaluation of anti-apoptosis protein, survivin-derived peptide vaccine in phase I clinical study for patients with advanced or recurrent breast cancer. J Transl Med 2008 10; 6:24.
14. Stauss H J, Thomas S, Cesco-Gaspere M, Hart D P, Xue S A, Holler A, King J, et al. WT1-specific T cell receptor gene therapy: improving TCR function in transduced T cells. Blood Cells Mol Dis 2008; 40(1):113-6.
15. Atanackovic D, Altorki N K, Cao Y, Ritter E, Ferrara C A, Ritter G, et al. Booster vaccination of cancer patients with MAGE-A3 protein reveals long-term immunological memory or tolerance depending on priming. Proc Natl Acad Sci USA 2008; 105(5):1650-5.
16. Odunsi K, Qian F, Matsuzaki J, Mhawech-Fauceglia P, Andrews C, Hoffman E W, et al. Vaccination with an NY-ESO-1 peptide of HLA class I/II specificities induces integrated humoral and T cell responses in ovarian cancer. Proc Natl Acad Sci USA 2007; 104(31):12837-42.
17. van Baren N, Bonnet M C, Dréno B, Khammari A, Dorval T, Piperno-Neumann S, et al. Tumoral and immunologic response after vaccination of melanoma patients with an ALVAC virus encoding MAGE antigens recognized by T cells. J Clin Oncol 2005; 23(35):9008-21.
18. Ajiro M, Katagiri T, Ueda K, Nakagawa H, Fukukawa C, Lin M L, et al. Involvement of RQCD1 overexpression, a novel cancer-testis antigen, in the Akt pathway in breast cancer cells. J Oncol 2009; 35(4):673-81.
19. Lin M L, Fukukawa C, Park J H, Naito K, Kijima K, Shimo A, et al. Involvement of G-patch domain containing 2 overexpression in breast carcinogenesis. Cancer Sci 2009; 100(8):1443-50.
20. Grigoriadis A, Caballero O L, Hoek K S, da Silva L, Chen Y T, Shin S J, et al. CT-X antigen expression in human breast cancer. Proc Natl Acad Sci USA 2009; 106(32):13493-8.
21. Frank B, Wiestler M, Kropp S, Hemminki K, Spurdle A B, Sutter C, et al. Association of a common AKAP9 variant with breast cancer risk: a collaborative analysis. J Natl Cancer Inst 2008; 100(6):437-42.
22. Lea I A, Richardson R T, Widgren E E, O'Rand M G: Cloning and sequencing of cDNAs encoding the human sperm protein, Sp17; Biochim Biophys Acta 1996, 1307:263-266. See also Chiriva-Internati M, Gagliano N, Donetti E, Costa F, Grizzi F, Franceschini B, et al. Sperm protein 17 is expressed in the sperm fibrous sheath. J Transl Med 2009 15; 7:61.
23. Nakazato T, Kanuma T, Tamura T, Faried L S, Aoki H, Minegishi T. Sperm protein 17 influences the tissue-specific malignancy of clear cell adenocarcinoma in human epithelial ovarian cancer. Int J Gynecol Cancer 2007; 17(2):426-32.
24. Gupta G, Sharma R, Chattopadhyay T K, Gupta S D, Ralhan R. Clinical significance of sperm protein 17 expression and immunogenicity in esophageal cancer. Int J Cancer 2007; 120(8):1739-47.
25. Grizzi F, Gaetani P, Franceschini B, Di Ieva A, Colombo P, Ceva-Grimaldi G, et al. Sperm protein 17 is expressed in human nervous system tumors. BMC Cancer 2006; 6:23.
26. Murakami, Y. et al. Toso, a functional IgM receptor, is regulated by IL-2 in T and NK cells. J. Immunol. 2012; 189, 587-597.
27. Lacy H M, Sanderson R D. Sperm protein 17 is expressed on normal and malignant lymphocytes and promotes heparan sulfate-mediated cell-cell adhesion. Blood 2001; 98(7):2160-5.
28. Li F Q, Han Y L, Liu Q, Wu B, Huang W B, Zeng S Y. Overexpression of human sperm protein 17 increases migration and decreases the chemosensitivity of human epithelial ovarian cancer cells. BMC Cancer 2009; 9:323.
29. Chiriva-Internati M, Grizzi F, Weidanz J A, Ferrari R, Yuefei Y, Velez B, et al. NOD/SCID tumor model for human ovarian cancer that allows tracking of tumor progression through the biomarker Sp17. J Immunol Methods 2007; 321(1-2):86-93.
30. Anderson K S. Tumor vaccines for breast cancer. Cancer Invest 2009; 27(4):361-8.
31. Kausar T, Ahsan A, Hasan M R, Lin L, Beer D G, Ralhan R. Sperm protein 17 is a novel marker for predicting cisplatin response in esophageal squamous cancer cell lines. Int J Cancer 2009 Aug. 14. [Epub ahead of print].
32. Chiriva-Internati M, Weidanz J A, Yu Y, Frezza E E, Jenkins M R, Kennedy R C, Cobos E, et al. Sperm protein 17 is a suitable target for adoptive T-cell-based immunotherapy in human ovarian cancer. J Immunother 2008; 31(8):693-703.
33. Chiriva-Internati M, Wang Z, Salati E, Wroblewski D, Lim S H. Successful generation of sperm protein 17 (Sp17)-specific cytotoxic T lymphocytes from normal donors: implication for tumour-specific adoptive immunotherapy following allogeneic stem cell transplantation for Sp17-positive multiple myeloma. Scand J Immunol 2002; 56(4):429-33.
34. Arora S, Matta A, Shukla N K, Deo S V, Ralhan R. Identification of differentially expressed genes in oral squamous cell carcinoma. Mol Carcinog 2005; 42(2):97-108.

What is claimed is:

1. A method for treating a breast cancer cell, a metastatic breast cancer cell, or a triple negative breast cancer cell comprising:
identifying a subject in need for treatment for a breast cancer cell, a metastatic breast cancer cell, or a triple negative breast cancer cell; and
administering a therapeutically effective amount of a formulation that leads to the presentation of an immunogenic SP17 protein or peptide antigen on an antigen presenting cell to activate T cells that are SP17-specific T cells, wherein the SP17-specific T cells impair the growth of the breast cancer cell, the metastatic breast cancer cell, or the triple negative breast cancer cell growth.

2. The method of claim 1, wherein the formulation further comprises an immunogenic SP17 protein or peptide antigen and an adjuvant.

3. The method of claim 1, wherein the formulation is defined further as an antigen presenting cell that has been pre-loaded with the isolated immunogenic SP17 protein or peptide.

4. The composition of claim 1, wherein the formulation is defined further as an antigen presenting cell that expresses an immunogenic SP17 protein or peptide and the antigen presenting cell is defined further as an autologous dendritic cell.

5. The composition of claim 1, wherein the isolated immunogenic SP17 protein or peptide antigen comprises a vaccine composition.

6. The method of claim 1, further comprising the steps of:
obtaining a sample from the subject; and
determining the presence or absence of a specific anti-SP17 immunoglobulins in the sample, wherein the presence of the specific anti-SP17 immunoglobulins in the sample indicating the subject is afflicted with or at least at risk of developing breast cancer.

7. The method of claim 1, wherein the sample is a blood sample.

8. The method of claim 1, further comprising obtaining naïve T cells from the subject, activating the naïve T cells in the presence of antigen presenting cells that present immunogenic SP17 under conditions that activate the T cells.

9. The method of claim 1, further comprising obtaining naïve T cells from the subject, activating the naïve T cells in the presence of antigen presenting cells that present immunogenic SP17 under conditions that activate the T cells in vitro and then introducing the activated T cells into the subject.

10. The method of claim 1, wherein the triple negative breast cancer is resistant to chemotherapeutic agents.

11. The method of claim 1, wherein the SP17 protein or peptide is loaded into a liposome for delivery into the antigen presenting cell, the SP17 protein or peptide is expressed in the antigen presenting cell by transient or stable transfection or the SP17 peptide is loaded directly onto MHC on the surface of the antigen presenting cell.

12. A method of treating a breast cancer cell, a metastatic breast cancer cell, or a triple negative breast cancer in a human subject comprising the steps of:
identifying a human subject in need of treatment for breast cancer cell, a metastatic breast cancer cell, or a triple negative breast cancer by detecting SP17; and
administering a therapeutically effective amount of a formulation that includes an isolated antigen presenting cells that is loaded with an immunogenic SP17 protein or peptide antigen capable of activating T cells to suppress the growth of the breast cancer cell, the metastatic breast cancer cell, or the triple negative breast cancer cell growth.

13. The method of claim 12, further comprising the step of obtaining a biological sample from the subject suspected of having SP17 and determining the present or absence of the SP17, wherein a decrease or the absence of SP17 in the biological sample following treatment is indicative that the treatment if effective.

14. The method of claim 12, wherein the antigen presenting cells is defined further as a dendritic cell.

15. The method of claim 12, wherein the sample is a blood sample.

16. The method of claim 12, further comprising obtaining naïve T cells from the subject, activating the naïve T cells in the presence of antigen presenting cells that present immunogenic SP17 under conditions that activate the T cells.

17. The method of claim 12, further comprising obtaining naïve T cells from the subject, activating the naïve T cells in the presence of antigen presenting cells that present immunogenic SP17 under conditions that activate the T cells and then introducing the activated T cells into the subject.

18. The method of claim 12, wherein the triple negative breast cancer is resistant to chemotherapeutic agents.

19. The method of claim 12, wherein the tumor cell or breast cancer is resistant tamoxifen.

20. The method of claim 12, further comprising the step of administering the therapeutic agent before, after, or concurrently as a combination therapy with one or more active agents that target at least one of breast cancer cells, metastatic breast cancer cells, or triple negative breast cancer cells.

21. The method of claim 12, wherein the triple negative breast cancer is resistant to chemotherapeutic agents.

22. The method of claim 12, further comprising the step of determining if the subject no longer responds to one or more chemotherapeutic agents prior to administering the antigen presenting cells that present SP17 to T cells.

23. A method of treating a cancer resistant to chemotherapy comprising the steps of:
identifying a subject having a breast cancer resistant to the chemotherapeutic agents or in need of increased effectiveness of the one or more chemotherapeutic agents, wherein the breast cancer is a triple negative breast cancer; and
administering a therapeutic agent carrier to the subject in an amount sufficient to suppress or inhibit the growth of breast cancer cells that express SP17 leading to an apoptosis, an arrested proliferation, or a reduced invasiveness of the breast cancer cells.

24. The method of claim 23, wherein the subject is further provided one or more chemotherapeutic agents comprise platinum drugs, carboplatin, tamoxifen, ER antagonists, or any combinations thereof.

25. The method of claim 23, wherein the inhibitor of SP17 is administered before, after, or concurrently as with the one or more chemotherapeutic agents.

26. The method of claim 23, wherein the breast cancer cells are triple negative breast cancer resistant to chemotherapeutic agents.

27. The method of claim 23, wherein the subject is a human.

28. A method for diagnosing breast cancer progression, prognosis, and resistance:
obtaining a biological sample from a patient suspected of having breast cancer;
detecting SP17 in the biological sample, wherein the presence of SP17 is indicative of the patient having breast cancer; and
administering a therapeutic agent that targets cells expressing SP17 in an amount effective to reduce the number of breast cancer cells in the patient.

29. The method of claim 28, wherein the therapeutic agent that targets cells expressing SP17 is provided before, after, or concurrently as a combination therapy with one or more chemotherapeutic agents that target at least one of breast cancer cells, metastatic breast cancer cells, or triple negative breast cancer cells.
